(19) United States
(12) Patent Application Publication
(10) Pub. No.: US 2026/0058016 A1
(43) Pub. Date: Feb. 26, 2026

(54) System

(71) Applicant: Softbank Group Corp., Tokyo (JP)

(72) Inventor: Kaho Mizunuma, Tokyo (JP)

(21) Appl. No.: 19/300,784

(22) Filed: Aug. 15, 2025

(30) Foreign Application Priority Data
Aug. 22, 2024 (JP) 2024-141282

(51) Int. Cl. G16H 50/20; G16H 40/67; G16H 80/00

(52) U.S. Cl. CPC G16H 50/20; G16H 40/67; G16H 80/00

(57) ABSTRACT

A system includes a processor that collects data representing activity levels, sleep patterns, and food intake from an information collection device attached to a dog, transmits the collected data to an analysis server, wherein the analysis server inputs the collected data into a generative artificial intelligence model and analyzes the data, transmits analysis results to a terminal and notifies a user, receives user input regarding questions or symptoms of the dog and generates advice using the generative artificial intelligence model, transmits the generated advice to the terminal for display to the user, and provides online consultation reservation with a veterinarian.

### CROSS-REFERENCE TO RELATED APPLICATION

This application is based on and claims priority under 35 USC 119 from Japanese Patent Application No. 2024-141282 filed on Aug. 22, 2024, which is incorporated by reference herein in its entirety.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional methods for managing a dog's health largely rely on subjective human observation and periodic veterinary visits, which are often insufficient for early detection of health issues. There is also a lack of efficient systems that can continuously collect, analyze, and provide actionable advice based on objective health data such as activity, sleep, and food intake. Furthermore, existing solutions do not adequately facilitate streamlined online consultations with veterinary professionals when abnormalities are detected.

### SUMMARY

The present invention provides a system comprising a processor that collects objective health data from a device attached to a dog, transmits the data to an analysis server, and utilizes a generative artificial intelligence model to analyze the data and generate advice for the user. The system further enables preprocessing by filtering abnormal values and supplementing missing data, identifies abnormalities in the dog's activity, sleep, and food intake, and presents the analysis results and advice to the user via a terminal. Additionally, the system allows users to easily reserve online consultations with veterinarians when necessary.

“Processor” means a hardware or software-based computational component capable of executing programmed instructions for controlling and managing system operations.

“Information collection device” means a hardware device, such as a smart collar, attached to a dog for the purpose of acquiring data related to the dog's activity, sleep patterns, and food intake.

“Activity levels” means a quantitative measure of the physical movement or exercise performed by a dog within a given period.

“Sleep patterns” means the measured duration, frequency, and quality of a dog's sleep as recorded by a monitoring device.

“Food intake” means the recorded amount and timing of food consumed by a dog, as gathered by sensors or user input.

“Analysis server” means a remote or network-connected computer system configured to receive, process, and analyze data sent from the information collection device or terminal.

“Generative artificial intelligence model” means an artificial intelligence system capable of analyzing input data and generating new outputs, such as analysis results and advice, based on learned patterns.

“Terminal” means a user-operated device, such as a smartphone or tablet, which is capable of receiving notifications and displaying information to the user.

“User” means an individual, typically the dog's owner or caregiver, who interacts with the system, receives notifications, and can input questions or symptoms.

“Advice” means information, recommendations, or specific instructions generated by the analysis server or artificial intelligence model for the user, based on processed data or user input.

“Veterinarian” means a qualified animal health professional who is available for consultation, including online appointments arranged through the system.

“Online consultation reservation” means a function within the system enabling the user to schedule and confirm remote appointments with a veterinarian via the terminal.

“Abnormal values” means data points or measurements which deviate significantly from the expected normal range, indicating potential errors or health concerns.

“Preprocessing” means a set of operations conducted on raw data prior to analysis, including filtering out abnormal values and supplementing missing data.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment; FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or”is employed to link three or more items in the present specification.

### First Exemplary Embodiment

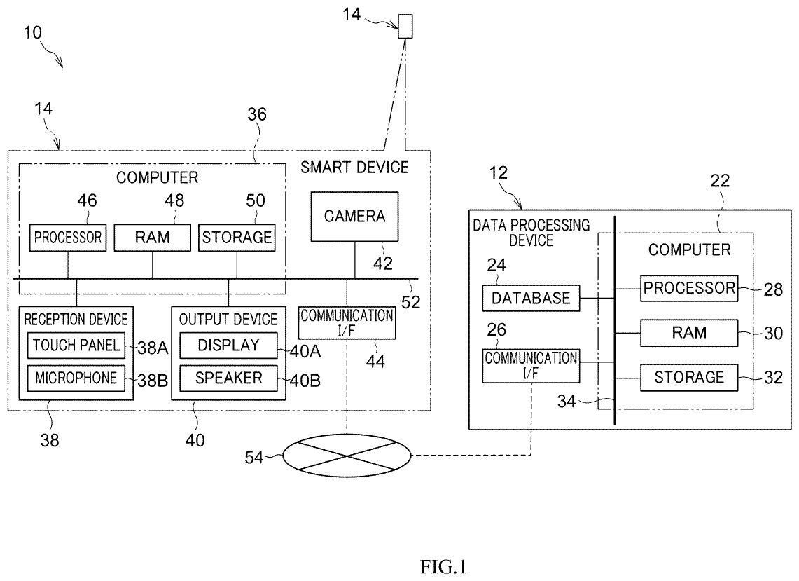

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

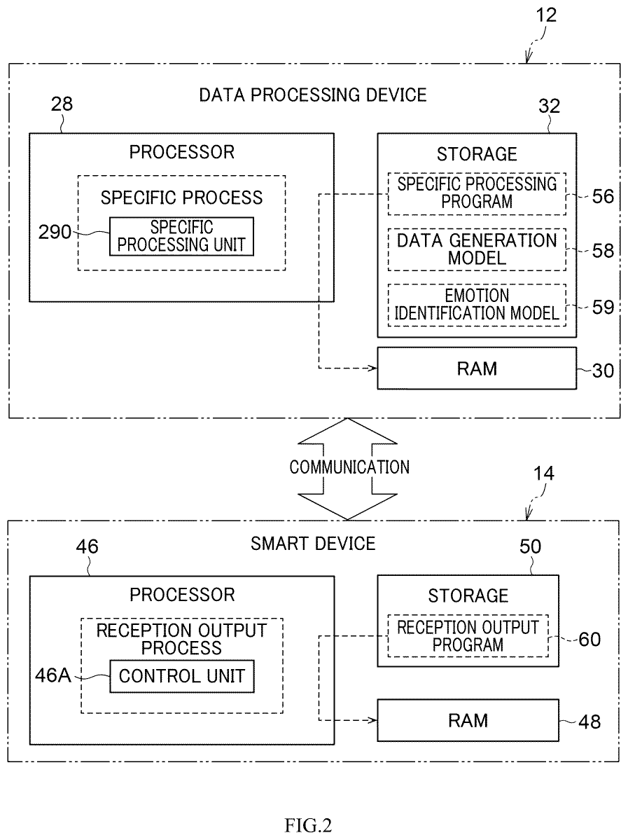

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventionally, systems for collecting and analyzing biological information of animals, such as health data from wearable devices, have suffered from insufficient integration among processes such as data collection, preprocessing, analysis, and provision of advice or consultation. This has resulted in cumbersome workflows for users, unreliable analysis due to lack of robust data preprocessing such as outlier detection and missing value supplementation, and limited user support for responding to detected abnormalities or symptoms. There is a need for an integrated solution that automates and streamlines the entire process, provides reliable data analysis and personalized advice, and facilitates expert consultation in a user-friendly manner.

The specific processing by the specific processing unit 290 of the data processing device 12 in Example 1 is realized by the following means.

The present invention provides a server comprising a processor configured to acquire biological information of an animal from a biological information collection device, perform data preprocessing including outlier detection and missing value supplementation, input the biological information into a generative artificial intelligence model via prompt sentences including analysis instructions, analyze the biological information, transmit analysis results and generated advice to a user device, accept inquiry or symptom-related input from the user, generate response information using the generative artificial intelligence model, provide reservation for remote consultation with a specialist, and integrally control the entire process by program control. This enables seamless and reliable management of animal health information from data collection through analysis, advice generation, user interaction, and expert consultation reservation, thereby improving the overall user experience and the usability of animal healthcare support.

The term “biological information” refers to data related to the physiological or behavioral state of an animal, including but not limited to activity level, sleep pattern, and food intake.

The term “biological information collection device” refers to an electronic apparatus attachable to an animal for the purpose of acquiring biological information, such as a wearable sensor or monitoring device.

The term “information processing device” refers to a general-purpose or dedicated computing apparatus that receives, processes, analyzes, and transmits data, typically implemented as a server.

The term “generative artificial intelligence model” refers to a computerized algorithm or system, such as a large language model, configured to perform reasoning, analysis, or generation of textual or analytical output based on input data.

The term “prompt sentence” refers to a structured or natural language input comprising instructions or queries, used to guide the operation of the generative artificial intelligence model.

The term “analysis processing” refers to operations conducted by the processor and/or artificial intelligence model for interpreting, identifying trends, anomalies, or generating insights from the biological information.

The term “user device” refers to a computing terminal, such as a smartphone, tablet, or computer, utilized by a user to interact with the system, receive transmissions, and submit inputs.

The term “user” refers to an individual operating the user device, typically responsible for the care of the animal.

The term “response information” refers to advice, recommendations, or answers generated by the generative artificial intelligence model in response to user-submitted inquiries or animal symptom reports.

The term “remote consultation reservation” refers to the process of scheduling an online or telecommunication-based appointment with a specialist, such as a veterinarian, facilitated by the system.

The term “specialist” refers to a professional qualified to provide expert consultation regarding animal health, such as a veterinarian.

The term “outlier detection” refers to the computational identification and handling of data points within the biological information that are inconsistent or deviate significantly from expected values.

The term “missing value supplementation” refers to the process of estimating and filling in absent or incomplete data entries within the biological information dataset.

The term “program control” refers to automated execution and management of the system's processes by software instructions within the processor.

One embodiment for implementing the present invention is described below.

The system may be constructed from three main hardware elements: a biological information collection device (such as a wearable sensor), a user device (such as a smartphone, tablet, or personal computer), and a processor (typically implemented as a server). The biological information collection device is attached to an animal, such as a dog, and is capable of automatically sensing and recording biological information including activity, sleep pattern, and food intake. An example of such hardware is a smart collar with motion sensors and wireless communication capability.

The terminal, which can be any general-purpose computing device operated by the user, is provided with an application for communicating both with the biological information collection device (via wireless methods such as Bluetooth or WiFi) and with the server (through the Internet using secure protocols such as HTTPS). The application is responsible for receiving the animal's biological information, transmitting this data to the server, displaying received notifications or advice to the user, facilitating user input related to symptoms or questions, and providing a reservation interface for remote consultation with a specialist.

The server, comprising at least one processor and appropriate storage, is equipped with software components that perform several sequential and integrated operations. Data analysis and processing routines are implemented using general-purpose programming languages such as Python, and data handling software libraries including Pandas and NumPy may be employed for preprocessing, such as outlier detection and missing value supplementation. The server communicates with an external generative artificial intelligence model, such as a large language model accessible via API, in order to process analysis instructions and generate advice or responses.

The process begins when the terminal receives the animal's biological information and transmits it to the server. The server preprocesses this data to remove outliers and supplement missing values, then assembles a prompt sentence containing analysis instructions. A typical prompt sentence might be:

“Analyze the following dog health data and identify any anomalies or trends: activity level, sleep hours, and food intake.”

The server then sends this prompt sentence and the biological information to the generative AI model. The generative AI model returns an analysis result, for example, “The dog's sleep time has dropped below average for the last three days,” which is further processed if necessary.

Following the data analysis, the server generates an additional prompt sentence to request tailored advice. An example of such a prompt sentence is:

“Given the reduced sleep time, generate clear advice for the animal owner.”

The generative AI model may return: “Limit the dog's activity for the coming days and observe whether sleep improves.”

The server transmits both the analysis result and the generated advice to the terminal. The terminal then notifies the user, displays the relevant information, and provides an interface for the user to submit questions or describe new symptoms such as, “The dog is coughing.” The terminal transmits this user input to the server, which then prepares another prompt sentence for the generative AI model, such as:

“The user reports that the dog is coughing. What advice should be provided?”

The generative AI model may respond: “If the cough continues for more than 24 hours, please schedule a veterinary consultation.”

When the user elects to reserve a remote consultation with a specialist, the terminal allows the user to select a desired date and time. The server processes this reservation request, manages schedules, confirms the booking, and sends a confirmation message to the user via the terminal.

Throughout all operations, the series of processes is performed under integrated program control within the server, with communications standardized through common Internet protocols and APIs. This configuration allows the invention to be realized efficiently using currently available hardware and software technologies and provides a seamless, reliable experience for animal health management from initial data collection through analysis, advice generation, user interaction, and specialist consultation reservation.

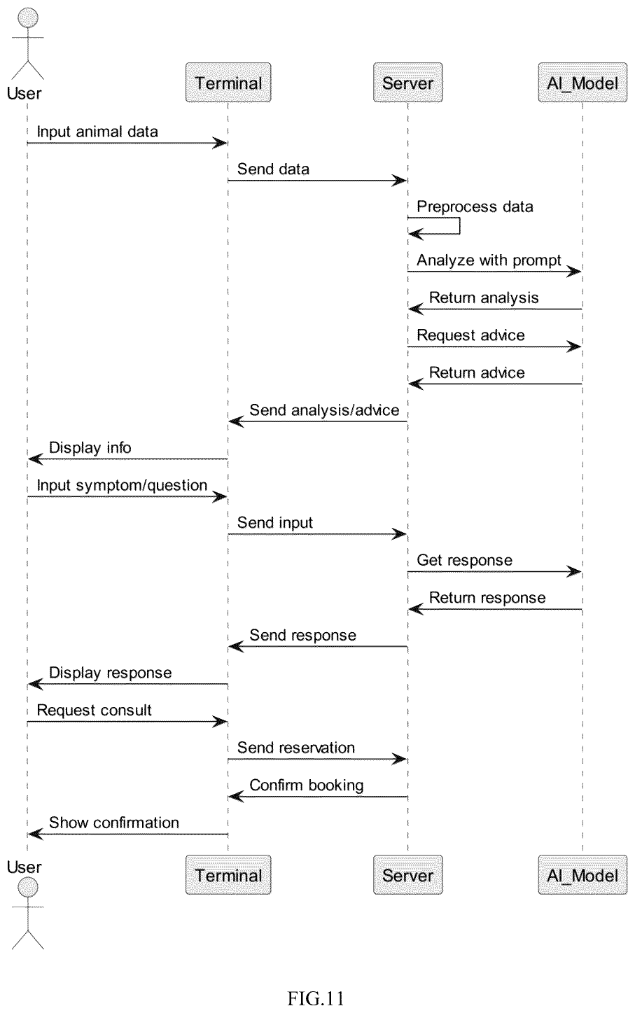

The following describes the processing flow using FIG. 11.

### Step 1

The terminal receives biological information from the biological information collection device attached to the animal via Bluetooth or WiFi. The input is raw sensor data including activity, sleep pattern, and food intake. The terminal stores this biological information in its local memory. The output is a dataset of biological information ready for transmission. For example, the terminal records that the animal has walked 3,200 steps, slept for 8 hours, and consumed 45 grams of food.

### Step 2

The terminal transmits the stored biological information to the server over the Internet using secure protocols, such as HTTPS. The input is the locally saved dataset of biological information. The terminal packages the data into a data structure, such as a JSON object, and sends it to the designated server endpoint. The output is the successful transmission of this biological information to the server.

### Step 3

The server receives the transmitted biological information from the terminal. The input is the received dataset containing activity, sleep pattern, and food intake. The server loads this dataset into a processing environment using data analysis libraries, such as Pandas. The output is a dataset available for preprocessing.

### Step 4

The server preprocesses the received biological information by performing outlier detection and missing value supplementation. The input is the dataset from the previous step. The server analyzes each data point, removes outliers (for example, extremely high activity values or invalid sleep readings), and fills in missing values based on previous trends or statistical means. The output is a cleaned and completed dataset. For example, if the food intake is missing for a particular day, the server substitues the average food intake from past data.

### Step 5

The server creates a prompt sentence that includes a specific analysis instruction and the cleaned biological information. The input is the preprocessed dataset. The server assembles a prompt, such as “Analyze the following dog health data and identify any anomalies or trends: activity level, sleep hours, and food intake.” The output is a textual prompt sentence ready for submission to the generative AI model.

### Step 6

The server submits the prompt sentence to the generative AI model for analysis. The input is the prompt sentence and cleaned biological data. The server sends this input via API to the generative AI model. The generative AI model analyzes the data based on the prompt and returns an analysis, such as “The dog's sleep duration has declined over the past three days.” The output is the textual analysis result from the generative AI model.

### Step 7

The server generates a follow-up prompt sentence requesting advice based on the analysis result. The input is the analysis result from the previous step. The server creates a prompt, for example, “Given the reduced sleep time, generate clear advice for the animal owner.” The server submits this prompt to the generative AI model, which responds with advice like “Limit the animal's activity for the next few days and observe whether sleep improves.” The output is the generated advice as a text message.

### Step 8

The server transmits both the analysis result and generated advice to the terminal. The input is the analysis and advice text generated by the generative AI model. The server packages this information and sends it to the terminal via secure transmission. The output is successful delivery of the analysis and advice to the terminal.

### Step 9

The terminal receives and displays the analysis and advice to the user using notifications and the main application interface. The input is the analysis and advice information from the server. The terminal updates its user interface, generates a notification, and presents the content visibly to the user. The output is that the user is informed of the animal's health analysis and recommended actions.

### Step 10

The user enters a question or symptom related to the animal's condition, such as “The dog is coughing,” into the app interface. The input is the user's text input. The terminal packages and sends this input to the server through an API call. The output is the successful transmission of the user's new input to the server.

### Step 11

The server receives the user input and creates a new prompt sentence for the generative AI model that incorporates the symptom or question. The input is the user's text input. The server forms a prompt such as “The user reports that the dog is coughing. What advice should be provided?” and submits this to the generative AI model. The model generates an appropriate response, for example, “If the cough continues for more than 24 hours, please schedule a veterinary consultation.” The output is the advice or answer text generated by the generative AI model.

### Step 12

The server sends the generated response to the terminal, which displays it to the user. The input is the response text created by the generative AI model. The terminal receives this information, updates the user interface, and displays the new advice or answer. The output is that the user receives further guidance or action steps for the animal's symptom.

### Step 13

The user initiates a remote consultation reservation request with a specialist using the terminal interface. The input is the user's selection of desired date and time for the reservation. The terminal sends this reservation request to the server. The output is the transmission of reservation details to the server.

### Step 14

The server processes the reservation request, manages the schedule, and confirms the booking with the user via the terminal. The input is the reservation request from the terminal. The server checks appointment availability, updates the scheduling system, and sends a confirmation to the terminal. The output is delivery of a confirmation message to the user, such as, “Your online consultation is scheduled for tomorrow at 10:00.”

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

In the field of animal healthcare management, it is difficult for users to continuously and accurately monitor the health status of animals, such as behavior, rest, and feeding patterns. Existing methods require manual data collection and analysis, which is time-consuming and may lead to delays in recognizing abnormal conditions. Furthermore, current systems do not adequately provide timely and personalized advice or incorporate the user's emotional state into the generated recommendations, and also lack a seamless interface for remote consultation reservations with medical professionals.

The specific processing by the specific processing unit 290 of the data processing device 12 in Application Example 1 is realized by the following means.

The present invention provides a server comprising a processor configured to acquire behavioral information, rest information, and feeding information from an information acquisition device worn by an animal, analyze the acquired information using a generative artificial intelligence model, generate advice according to both the animal's state and the user's emotional status, transmit the analysis and advice to a display device, receive input or inquiry from the user, create a prompt sentence from the user's inquiry, the emotional state, and the acquired information, generate further advice using the generative artificial intelligence model based on the prompt sentence, and provide a remote consultation reservation system with a medical professional via a communication network. This enables automatic and real-time monitoring of an animal's health status, provides timely and personalized recommendations to the user, reflects the user's emotions in the output advice, and facilitates immediate and convenient arrangements for remote consultations.

The term “processor” refers to a hardware or software component capable of executing instructions to process data and control operations within a system.

The term “information acquisition device” refers to a hardware component attachable to an animal that collects biological and activity data, such as behavioral, rest, and feeding information.

The term “behavioral information” refers to data indicating the movements, activities, or actions performed by an animal, such as walking, running, or other detectable behaviors.

The term “rest information” refers to data indicating the sleeping patterns, rest durations, or periods of inactivity of an animal.

The term “feeding information” refers to data indicating the quantity, timing, or frequency of food or water intake by an animal.

The term “computing device” refers to a general-purpose or specialized electronic device capable of receiving, processing, and transmitting data according to programmed instructions.

The term “generative artificial intelligence model” refers to an artificial intelligence model that processes input data and automatically generates analytical results, advice, or recommendations, typically based on machine learning or deep learning algorithms.

The term “analysis result” refers to conclusions, evaluations, or alerts generated from the acquired animal data after processing by the generative artificial intelligence model.

The term “display device” refers to any electronic device or interface capable of presenting information, notifications, or advice to a user visually or audibly.

The term “user” refers to a person who operates or interacts with the system, typically the owner or caretaker of the animal.

The term “inquiry” refers to any question, request for advice, or report of symptoms submitted by the user regarding the animal's condition.

The term “advice information” refers to recommendations, suggestions, or guidance generated by the system based on analysis of animal data and user interaction.

The term “emotional state” refers to a detected or inferred psychological condition of the user, such as stress, anxiety, or calmness, determined through sensor input, voice, facial expression analysis, or direct user input.

The term “remote consultation reservation” refers to a booking system that allows the user to schedule an online or remote appointment with a medical professional, such as a veterinarian, via a communication network.

The term “prompt sentence” refers to a structured input sentence or set of instructions formed from user inquiries, emotional state, and acquired data, which is fed to the generative artificial intelligence model to produce customized advice.

The term “outlier detection” refers to a data processing technique for identifying abnormal, inconsistent, or erroneous values within the acquired information.

The term “missing value imputation” refers to a process for estimating and filling in absent or incomplete data entries within the acquired information.

The term “period-based health analysis result” refers to a summary or evaluation of the animal's health status generated for a specified time period, such as daily or weekly, based on accumulated data.

One embodiment for implementing the invention will now be described in detail. The present invention may be carried out by integrating general-purpose or specialized hardware components, such as an information acquisition device (for example, a smart wearable collar), a terminal device (such as a smartphone, tablet, or general-purpose mobile device), and at least one server (cloud server or dedicated computer). In addition, software components may include: mobile operating systems (such as Android or iOS), data communication protocols (such as Bluetooth or WiFi module software), and artificial intelligence frameworks (such as TensorFlow or PyTorch), as well as web server and API software.

The information acquisition device, worn by an animal, is equipped with sensors capable of collecting behavioral information (such as motion or acceleration data), rest information (such as sleep period, detected via inactivity and heart rate variation), and feeding information (such as intake detected via motion near a feeding bowl or weight sensor). For example, the information acquisition device may be implemented using a combination of accelerometers, gyroscopes, heart-rate sensors, or similar commercially available components. The device periodically transmits collected data to the terminal device using a wireless protocol such as Bluetooth or WiFi.

The terminal device receives the transmitted animal data, temporarily stores it in memory, and preprocesses it as needed (for example, data format conversion, optional anonymization of user identifiers, and preliminary validation). The terminal device establishes a secure internet connection and transmits the collected data packages to a server. This transmission may be performed using HTTPS POST requests via a RESTful API. The terminal device may also prompt the user to provide input regarding the animal's symptoms, inquiries, or observations, and may further collect the user's emotional state using input methods such as voice input, facial recognition, or in-app questionnaires.

The server receives the uploaded data and stores it in a data storage system (such as a cloud database). The server conducts preprocessing steps, such as outlier detection and missing value imputation, using standard data cleaning scripts (implemented, for example, in Python). The preprocessed data is then provided as input to a generative artificial intelligence model implemented via a machine learning framework (such as a TensorFlow or PyTorch model).

The generative AI model analyzes the comprehensive animal data, detects abnormalities or trends, and generates a summary or specific advice in natural language.

Depending on the user's emotional state—derived from voice data, facial image analysis, or explicit responses—the content and tone of the advice may be dynamically adjusted. The server then transmits the advice and analytic results to the terminal device for presentation to the user. The user is notified of relevant warnings or suggestions via system notifications or in-app alerts, and may review temporal health analysis reports (e.g., daily or weekly summaries) through interactive dashboards within the application.

In addition, the system provides a remote consultation reservation function. The user can request an appointment with a medical professional through the application interface, and the server processes and synchronizes the reservation data with the medical institution's system using a communication protocol such as WebSocket or REST API.

Specific examples of how the system may be used include the following:
The user equips the animal with the information acquisition device. The terminal collects behavioral and rest information over several days.
The server receives the data and identifies a pattern of reduced activity and excessive rest. The generative AI model generates advice such as: “Your animal's activity is notably low this week. Please consider extending daily walks or stimulating more playtime.”
If the user reports an inquiry such as “My animal is not eating well” and sounds anxious, the terminal collects and analyzes this input and communicates it to the server, which generates advice like: “We understand your concern. Monitor your animal's feeding and, if the issue persists, consult a medical professional.”
The user can press a button in the application to book a telemedicine appointment, and the reservation details are immediately shared with the medical provider.

Example prompt sentences fed to the generative AI model include:

“As a pet health assistant, given: activity 1200 steps/day, sleep 16 hours/day, food intake 400 g/day, generate advice for the owner regarding potential concerns and action steps.”

“Owner reports: ‘My animal is coughing.’ With recent low activity and unchanged appetite, provide specific advice and recommend if veterinary consultation is necessary.”

“Owner emotion: anxious. Compose supportive advice, acknowledge their concerns, and suggest actionable next steps.”

Through such hardware and software integration, the invention can be practiced to enable real-time, automated, and emotionally-aware animal health management, prompt delivery of personalized professional advice, and seamless arrangements for remote consultations with medical professionals.

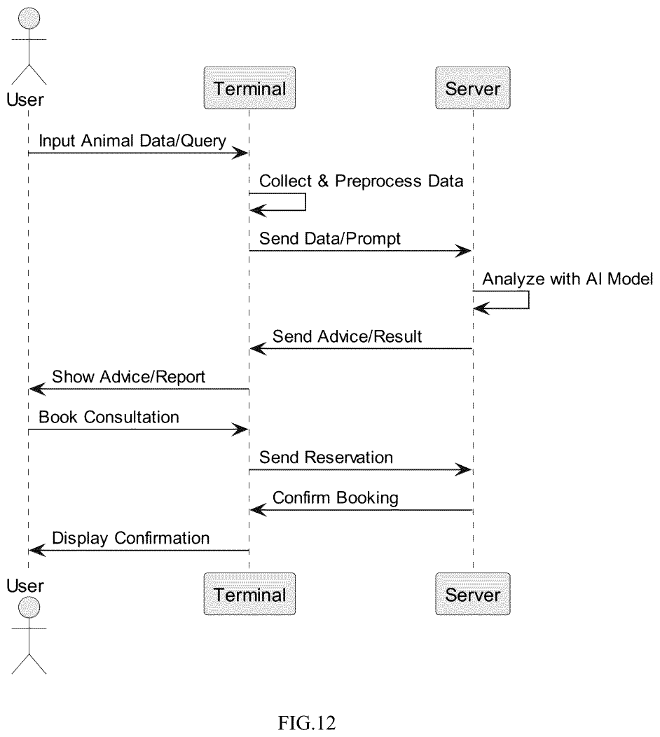

The following describes the processing flow using FIG. 12.

### Step 1

The terminal establishes a wireless connection (via Bluetooth or WiFi) with the information acquisition device attached to the animal. The terminal requests and receives behavioral information, rest information, and feeding information from the acquisition device at set intervals. The input is raw sensor data from the device, and the output is structured animal data stored locally on the terminal. The terminal converts sensor signals into a formatted data package and timestamps the records.

### Step 2

The terminal preprocesses the collected animal data, including formatting, optional anonymization, and preliminary validation. The input is the structured animal data, and the output is a validated dataset ready for transmission. The terminal cleans the records by checking for data completeness and applying basic corrections.

### Step 3

The terminal transmits the preprocessed data to the server via a secure internet connection (such as HTTPS POST to a REST API). The input is the validated dataset, and the output is the successful delivery of animal data to the server. The terminal packages and compresses the data, then sends it to the designated server endpoint.

### Step 4

The server receives the animal data from the terminal and stores it in a database. The input is the uploaded animal data, and the output is a persistent data record on the server. The server parses the uploaded file, checks the integrity of the data, and inserts the structured information into a database system.

### Step 5

The server preprocesses the data by performing outlier detection and missing value imputation using predefined statistical rules. The input is the raw animal data from the database, and the output is a cleaned and normalized dataset. The server runs preprocessing scripts that filter abnormal values and estimate missing entries through interpolation or statistical methods.

### Step 6

The server inputs the preprocessed dataset into a generative AI model for analysis. The server uses the model to detect patterns, identify abnormalities related to animal health, and generate a natural language summary. The input is the cleaned dataset, and the output is an analysis result with corresponding advice. The server converts the AI model output to text suitable for user communication.

### Step 7

The server transmits the generated analysis result and advice to the terminal, using push messaging or notification services. The input is the analytic result and advice text, and the output is a successful notification sent to the terminal. The server packages the advice in a notification format and initiates delivery.

### Step 8

The terminal receives the server's advice and presents it to the user as a notification and graphical dashboard. The input is the advice and analytic result, and the output is the updated application interface and visual charts for the user. The terminal also logs user actions such as notification taps or report viewing for further reference.

### Step 9

The user reviews the advice and can respond with feedback, questions, or additional information about the animal's condition, including inputting emotional state through voice or questionnaires. The input is the presented advice/results, and the output is user input data (question, inquiry, or emotional feedback) captured by the terminal. The user interacts with input forms or voice interfaces on the app.

### Step 10

The terminal collects the user's responses and emotional state, and assembles a prompt sentence that combines animal data, user inquiry, and emotional profile. The input is the user's input and stored animal data, and the output is a constructed prompt for the generative AI model. The terminal formats the combined information into a structured prompt for the server.

### Step 11

The terminal transmits the prompt sentence and related user input to the server for further processing and advice generation. The input is the prompt sentence, and the output is the successful receipt of the prompt by the server. The terminal sends this prompt to the server as a new advisory request.

### Step 12

The server processes the prompt sentence using the generative AI model, taking into account the latest animal data, user inquiry, and the user's emotional state. The input is the received prompt sentence, and the output is tailored advice or recommendations. The server generates emotionally-adaptive advice and prepares it for delivery.

### Step 13

The server transmits the tailored advice to the terminal, which displays the response to the user. The input is the advice generated by the AI model, and the output is the updated user interface showing individualized recommendations. The terminal uses notification and display logic to inform the user.

### Step 14

The user may initiate a remote consultation reservation with a medical professional using the terminal application. The input is the user's selection of appointment details, and the output is a reservation request sent to the server. The user interacts with the reservation interface.

### Step 15

The server receives the reservation request and synchronizes the booking with the external medical institution's system (using, for example, API or real-time communication protocol). The input is the reservation request, and the output is a confirmation or appointment record accessible to both the user and the provider. The server updates records and sends a confirmation notification.

### Step 16

The server periodically generates period-based health analysis results (such as daily or weekly reports) using accumulated animal data, and transmits these reports to the terminal. The input is the accumulated animal data over a period, and the output is a summary report or visualization for user review. The server compiles and formats the report for delivery, and the terminal notifies the user and displays the report for ongoing health monitoring.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

In conventional systems for managing companion animal health, data collection is limited to animal activity, sleep, and food intake information, and user emotional states are not considered in the generation and delivery of advice. As a result, the advice provided may not be suitable to the user's emotional context, reducing efficacy and user compliance. Furthermore, conventional systems often lack integrated means for online communication and reservation with specialists, such as veterinarians, requiring users to utilize multiple platforms and decreasing system convenience and connectedness.

The specific processing by the specific processing unit 290 of the data processing device 12 in Example 2 is realized by the following means.

The present invention provides a server comprising a processor configured to collect biometric activity information from a biometric information collection device, preprocess the information by removing outliers and completing missing values, analyze the preprocessed information using a generative information processing model, generate behavioral guidance information based on analysis results, acquire the user's emotional state using voice and image recognition, adjust advice in accordance with the user's emotional state, automatically generate a prompt sentence for the generative information processing model using both biometric and emotional state data, and manage online specialist reservation and communication. This enables effective and context-aware companion animal health management, improves the appropriateness and user-friendliness of provided advice, and facilitates seamless reservation for remote consultation with specialists.

The term “biometric activity information” refers to data related to physiological activities of a living organism, such as movement, sleep patterns, and food intake, which are collected by a sensor device attached to the organism.

The term “biometric information collection device” refers to a hardware apparatus equipped with one or more sensors that is attached to an organism for the purpose of measuring and transmitting biometric activity information.

The term “data processing apparatus” refers to an electronic device or server comprising a processor configured to receive, store, process, analyze, and communicate information obtained from external sources, such as biometric information collection devices and terminal devices.

The term “data preprocessing” refers to computational procedures performed on raw data to improve its quality and suitability for analysis, including outlier removal and missing value imputation.

The term “generative information processing model” refers to an information processing system or algorithm capable of analyzing input data and generating descriptive or prescriptive output, such as analysis results, recommendations, or advice, based on learned patterns.

The term “behavioral guidance information” refers to information comprising recommendations, instructions, or advice generated by the system to guide user actions relating to the health or well-being of an organism.

The term “terminal device” refers to a user-operated electronic device, such as a smartphone or tablet, configured to receive, transmit, display, and input data in connection with the system.

The term “remote specialist communication” refers to an interaction method enabling users to consult with specialists, such as healthcare professionals, using telecommunication networks to exchange information or advice.

The term “reservation management” refers to processes or systems that allow users to schedule, confirm, and manage appointments or consultations with specialists via the system.

The term “emotional state information” refers to data indicating the psychological or affective status of a user, derived from input such as voice or image recognition.

The term “prompt sentence” refers to an input sentence or instruction automatically generated by the system to guide the operation of the generative information processing model, typically incorporating context from biometric activity information and user emotional state information.

An embodiment of the present invention provides a system for companion animal health management that integrates biometric activity information collection, data preprocessing, analysis using a generative information processing model, and behavioral guidance adjusted according to user emotional state, along with the capability for remote specialist consultation reservation.

The user attaches a biometric information collection device, for example a smart collar equipped with movement, temperature, and feeding sensors, to a companion animal. The terminal device, such as a smartphone or tablet, communicates with the biometric information collection device via Bluetooth or WiFi using protocols such as Bluetooth Low Energy or IEEE 802.11, and periodically retrieves biometric activity information including activity levels, sleep duration, and food intake records.

The terminal device uses built-in hardware such as a microphone and camera to capture the user's voice and facial images when prompted by the application. The terminal device processes these media inputs using emotion recognition software, for example, general-purpose SDKs or publicly available AI platforms for voice recognition and facial emotion analysis, and extracts emotional state information about the user.

The terminal device transmits both the biometric activity information and the user emotional state information to the server via a secure internet connection, typically using the HTTPS protocol and REST-style APIs.

The server, embodied as a data processing apparatus, uses software libraries such as pandas and scikit-learn on a general-purpose server or cloud computing instance to perform data preprocessing. This includes the automatic removal of outlier records and the completion of missing values via statistical estimation.

The server formats the preprocessed data and emotional state information into a prompt sentence, for example:

“Analyze the dog's activity data and generate advice if an anomaly is detected.”

or

“Consider the user's current emotional state when generating advice for animal care.”

The server inputs this prompt sentence, along with the associated data, into a generative information processing model such as a large language model trained for data analysis and advice generation. Well-known generative AI platforms or models, such as those accessible through commercial APIs or self-hosted frameworks, can be used.

Based on the analytical results and the emotional state information, the server generates behavioral guidance information. If, for example, a reduction in the animal's activity is detected, and if the user's emotional state is assessed as stressed, the generated guidance could be:

“The dog's activity level has decreased. We recommend increasing the duration of daily walks. Since you are experiencing stress, try to find relaxing moments together with your dog.”

The server then transmits the generated behavioral guidance to the terminal device, which displays the advice within the application UI and notifies the user using the notification system of the device (e.g., Android or iOS push notifications).

If the user enters an inquiry, such as “My dog is coughing,” via the application interface, the terminal device transmits the inquiry data to the server. The server generates a specific prompt sentence such as:

“The user reported: ‘My dog is coughing.’ Generate advice and recommend online consultation if needed.”

The server supplies the prompt and data to the generative information processing model, which outputs personalized response information. The terminal device displays this response and, if appropriate, provides a reservation interface to schedule an online consultation with a remote specialist. The server manages reservation data, confirms appointments, and notifies the user through the terminal device.

The above embodiment enables practical and adaptive management of animal and user health status, improves the suitability of the provided advice, and offers an integrated way to manage remote specialist consultations. This system can be implemented using commonly available server infrastructure, general-purpose mobile devices, and widely used AI platforms for natural language and emotion analysis.

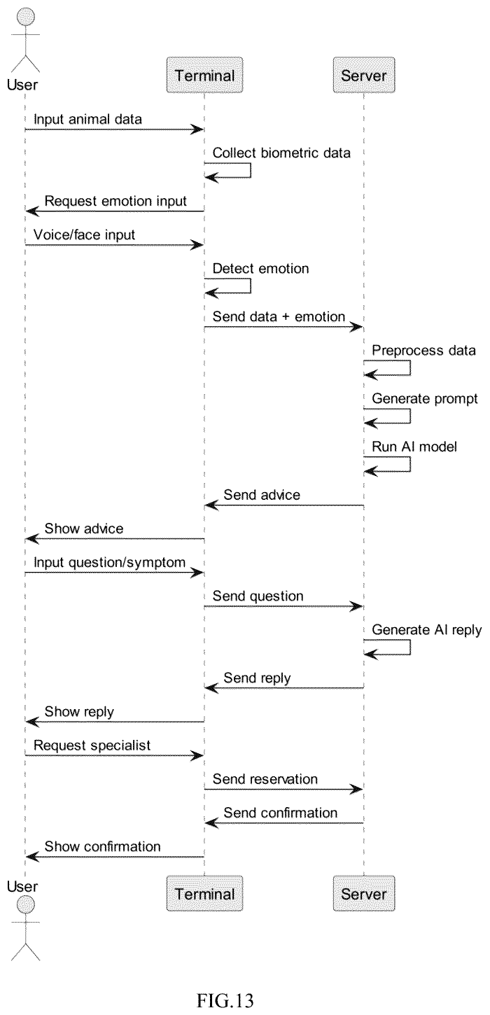

The following describes the processing flow using FIG. 13.

### Step 1

The terminal establishes a wireless connection (using Bluetooth or WiFi) with the biometric information collection device attached to the companion animal. As input, the terminal requests activity, sleep, and food intake data from the device. The device transmits the raw sensor data to the terminal. The terminal stores these records in a local database.

### Step 2

The terminal prompts the user to provide emotional input by capturing their voice through the built-in microphone and taking a facial image with the camera. The inputs are the user's voice and facial image data. The terminal processes this data using emotion recognition software and outputs the detected emotional state, such as “stressed” or “neutral.” The emotional state is saved for further processing.

### Step 3

The terminal combines the collected biometric activity data and emotional state information. As input, the terminal uses the sensor data and emotional state output from Steps 1 and 2. The terminal formats the combined data into a standardized data package (e.g., JSON format) and transmits this as output to the server using a secure internet connection.

### Step 4

The server receives the transmitted data package from the terminal as input. The server stores the data temporarily and initiates a data preprocessing routine. In this step, the server uses data processing software (e.g., pandas and scikit-learn) to filter outliers and fill missing values in the biometric data, outputting a clean data set ready for analysis.

### Step 5

The server prepares a prompt sentence using the preprocessed biometric data and emotional state information. The input in this step is the cleaned data set and emotional state. The server generates a text-based instruction such as “Analyze the dog's activity data considering the user's current emotional state.” The output is a prompt sentence to be used for the generative AI model.

### Step 6

The server inputs the prompt sentence and prepared data into the generative AI model. As input, the model receives the stored data and prompt sentence. The generative AI model analyzes the data and generates behavioral guidance information as output, for example, “The dog's activity level is low. Recommend more frequent walks. As the user is stressed, suggest relaxation activities together.”

### Step 7

The server sends the generated behavioral guidance information to the terminal as output. As input, the terminal receives the advice message from the server. The terminal uses the device's notification system to alert the user and updates the application UI to display the new guidance.

### Step 8

The user submits questions or symptom descriptions through the app's form. The input is user-typed text, such as “My dog is coughing.” The terminal sends this inquiry to the server as output.

### Step 9

The server receives the user inquiry and prepares a new prompt sentence, such as “The user reported: ‘My dog is coughing.’ Generate advice and recommend online consultation if appropriate.” Using the prompt and the inquiry as input, the server utilizes the generative AI model to generate personalized advice and outputs it.

### Step 10

The server transmits the personalized advice to the terminal. The terminal receives this advice and displays it in the application's consultation result screen. If the advice includes a recommendation for specialist consultation, the terminal activates the reservation function for online communication with a remote specialist.

### Step 11

The user interacts with the reservation interface on the terminal to select a desired time and specialist for remote consultation. The input is the chosen date, time, and specialist. The terminal sends the reservation request to the server as output.

### Step 12

The server receives the reservation request. The server validates the availability, confirms the reservation, and generates a confirmation notification as output. The server transmits the confirmation to the terminal, which then notifies the user and displays the reservation details on the device.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional animal care systems primarily focus on collecting and analyzing health-related data of animals, such as activity, sleep, and feeding patterns. However, these systems lack the capability to take into account the emotional state of the user when providing advice, which can result in less effective support and lower user satisfaction. Furthermore, the process for users to communicate symptoms or concerns and to arrange timely consultation with professionals such as veterinarians is often cumbersome and inefficient. As a result, there is a need for a system that can provide more personalized advice by considering both animal health data and user emotional state, while also streamlining the consultation reservation process.

The specific processing by the specific processing unit 290 of the data processing device 12 in Application Example 2 is realized by the following means.

The present invention provides a server comprising a processor configured to collect biological activity information, sleep information, and feeding information from a data collection device attached to an animal, to obtain the emotional state information of a user, to generate input data for use by a generative artificial intelligence model, to analyze the input data and generate advice information reflecting the user's emotional state, to provide notification and display of the analysis result and advice to the user, to receive inquiries or animal symptom information from the user, to generate response information using the generative artificial intelligence model, and to facilitate reservation for remote communication with a professional. This enables the provision of personalized advice by integrating both animal health data and user emotion, and also simplifies the process for arranging timely consultations with professionals.

The term “biological activity information” refers to data representing the physical movements or activity levels of an animal, including but not limited to walking, running, and other exercise activities.

The term “sleep information” refers to data related to the duration, quality, or patterns of sleep of an animal.

The term “feeding information” refers to data indicating the amount, frequency, or type of food consumed by an animal.

The term “data collection device” refers to a hardware apparatus attached to an animal that is configured to collect at least one of biological activity information, sleep information, or feeding information.

The term “information processing device” refers to a computing apparatus or server that processes data received from the data collection device and the user's input.

The term “communication apparatus” refers to a hardware or software component capable of wireless or wired data transmission between devices in the system.

The term “user's emotion information” refers to data representing the emotional state of the user, which may be determined based on audio, video, biometric, or textual input.

The term “terminal device” refers to a user-operated device, such as a smartphone or tablet, configured to send, receive, and display information within the system.

The term “generative artificial intelligence model” refers to an algorithm or set of algorithms employing artificial intelligence technology that is capable of generating analysis results, advice, or responses based on input data.

The term “advice information” refers to automatically generated recommendations, instructions, or notifications for the user based on the results of data analysis.

The term “analysis result” refers to output produced by the generative artificial intelligence model after processing the input data, including but not limited to health assessments or condition alerts.

The term “response information” refers to information generated by the generative artificial intelligence model in reply to user inquiries or animal symptom input.

The term “professional” refers to a person engaged in a specialized occupational role, such as a veterinarian or animal health care provider, who may be consulted within the system.

The term “communication reservation” refers to a system-provided capability allowing users to schedule and manage remote consultations or communications with a professional.

### Embodiment for Implementing the Invention

The present invention may be embodied as a system integrating a data collection device, terminal device, information processing device, and a generative artificial intelligence model to enable comprehensive care of an animal, considering both health data and the emotional state of the user. The system combines hardware and software components to collect, process, and analyze various data points and then provide personalized advice and consultation arrangements to the user.

In a typical embodiment, the system utilizes a wearable data collection device secured to the animal, such as a smart collar. The data collection device may include sensors for measuring biological activity information (such as step count and movement), sleep information (duration and quality), and feeding information (amount and timing). These sensors may be realized using commercially available microcontrollers and MEMS sensors.

The terminal device, which may be a mobile communication device such as a smartphone or tablet, is operated by the user. The terminal device is equipped with a communication apparatus (for example, Bluetooth Low Energy or Wi-Fi) for receiving data from the data collection device. Furthermore, the terminal device includes a camera and microphone to collect the user's emotion information by capturing facial expressions and voice data. This input may be processed by emotion recognition software, such as cloud-based recognition and speech-to-text APIs.

The terminal device formats the collected health and emotion information and transmits it through the internet to the information processing device, which is implemented as a server. The server may be hosted on a general-purpose computing platform or a cloud service. The information processing device is programmed with logic to preprocess the received data, including data cleaning, abnormal value detection, and missing data supplementation. Algorithms may be implemented using standard software libraries such as Python pandas or machine learning packages.

The server is further configured to construct input data for the generative artificial intelligence model. The generative AI model may be implemented as a cloud-based service (for example, a model similar to GPT-4 or any advanced language model). The server submits a prompt sentence to the model, encompassing both the animal's latest health data and the user's emotion information, in natural language. In response, the generative AI model analyzes the data and generates advice information that considers both aspects.

The server then transmits the advice information and analysis results to the terminal device. The terminal device displays the advice to the user either as a push notification or as content on a dedicated section of the application. All advice may be stored in the history log for future reference.

The system further supports input from the user, allowing the user to submit questions or report symptoms concerning the animal using a graphical interface of the terminal device. The input is sent to the server, which uses the generative artificial intelligence model to generate a response. The response can include instructions, information about potential health issues, or a recommendation to book a remote communication session with a professional, such as a veterinarian.

The server manages a reservation system for remote communication, enabling the user to choose and confirm an appointment time for consultation with the professional.

Confirmation of appointments is communicated to both the user and the professional through the terminal device.

### Concrete Examples

The user interacts with the app, and the terminal device collects the following data: “The dog's activity decreased from 4000 steps to 2300 steps in the past two days; the user appears stressed based on facial recognition.”
Using a prompt sentence such as, “Generate advice for an owner who is stressed and whose dog has reduced activity,” the server obtains a personalized recommendation like, “You and your dog may benefit from a longer walk together this evening.”
If the user inputs, “My dog has a persistent cough,” a prompt such as, “Suggest actions for an owner reporting a persistent cough in their dog,” is generated, and the system may reply, “Monitor the cough and book an online consultation if symptoms do not improve.”

### Examples of Prompt Sentences Sent to the Generative AI Model

“Analyze the following information: The dog's step count is 1500 today, sleep duration is 6 hours, and feeding is 200 grams. The user appears unhappy. Generate advice for the user.”
“Determine if the user is experiencing stress based on their voice and facial data and provide appropriate advice.”
“Suggest the next steps for a dog that has not eaten for two days.”
“Please provide a supportive message for a user who feels sad and whose dog shows decreased sleep quality.”

Through this structure, the invention enables seamless integration of animal health monitoring, user emotion recognition, automated advice generation, and remote professional consultation within a unified system.

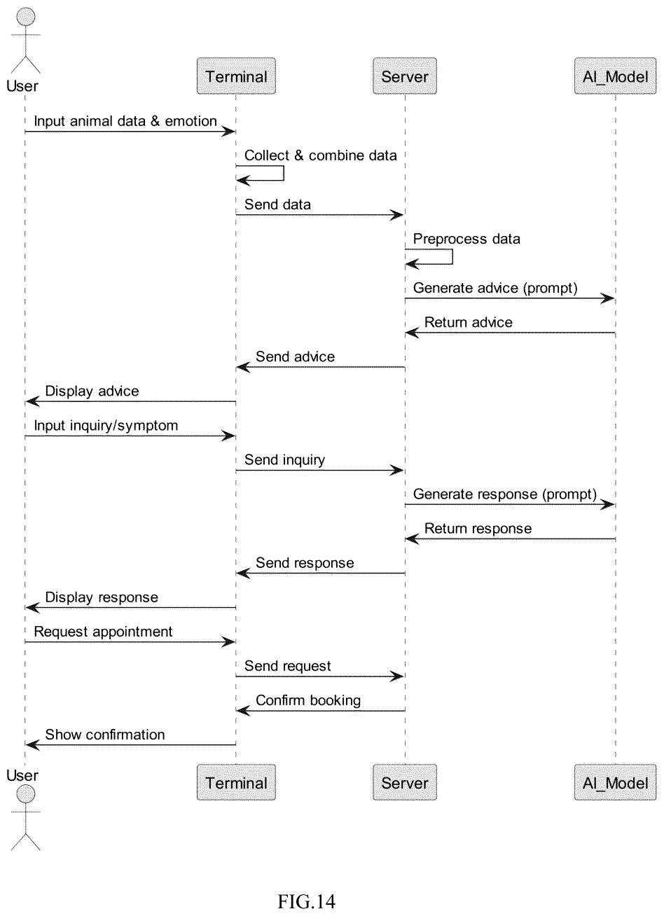

The following describes the processing flow using FIG. 14.

### Step 1

The terminal establishes a wireless connection (such as Bluetooth or Wi-Fi) with a data collection device attached to the animal. The terminal requests and receives biological activity information, sleep information, and feeding information from the sensors on the device.

Input: Signals and raw sensor data from the data collection device.

Processing: The terminal parses the received data, converts it into structured health metrics, and stores the information locally.

Output: Structured health data, including activity level, sleep duration, and feeding quantity, stored on the terminal.

### Step 2

The terminal collects user emotion information by activating its camera and microphone. The terminal captures facial images and records voice data from the user during interaction with the application.

Input: Image and audio data from the terminal's camera and microphone.

Processing: The terminal processes this data locally or sends it to an emotion recognition service, which analyzes facial expressions and vocal features to determine the user's emotional state.

Output: Processed emotion data, such as emotion category (e.g., “stressed”, “happy”) and associated confidence score.

### Step 3

The terminal combines the structured health data of the animal with the processed user emotion data into a single data payload. The terminal then sends this payload securely over the internet to the server.

Input: Structured health data and processed emotion data collected in Steps 1 and 2.

Processing: The terminal serializes the data, encrypts it, and transmits it via HTTPS or other secure protocols to the server's endpoint.

Output: Data payload containing animal health metrics and user emotion information transmitted to the server.

### Step 4

The server receives the data payload from the terminal. The server then preprocesses the received data by performing abnormal value determination and data completeness checks. Input: Animal health metrics and user emotion information from the terminal.

Processing: The server runs algorithms to filter out abnormal values, fill missing data entries (using interpolation or past data), and normalize the information for further analysis.

Output: Cleaned and complete dataset of animal health and user emotion information.

### Step 5

The server generates a prompt sentence based on the cleaned health data and user emotion information. The server submits the prompt to a generative AI model for analysis.

Input: Normalized and complete dataset of animal health and user emotion information.

Processing: The server constructs a natural language prompt, sends it to the generative AI model (such as a large language model), and awaits the response.

Output: Context-aware advice text and an analysis result generated by the generative AI model.

### Step 6

The server transmits the advice and analysis results to the terminal for the user.

Input: Advice text and analysis result from the generative AI model.

Processing: The server packages the advice and results into a message, and sends it to the user's terminal device using a secure communication method.

Output: Advice message and analysis result delivered to the terminal.

### Step 7

The terminal receives the advice and analysis result from the server and notifies the user. The terminal displays the content in a dedicated section of the application or as a push notification.

Input: Advice message and analysis result from the server.

Processing: The terminal decodes and renders the advice, updates the app's user interface, and logs the advice history.

Output: Visible display of advice and analysis result on the terminal's screen for the user.

### Step 8

The user inputs a follow-up question, symptom report, or request for professional consultation through a form or interactive feature in the application.

Input: User's text or voice input describing a concern or question about the animal. Processing: The terminal interprets or transcribes the input and prepares it for transmission.

Output: User inquiry, symptom description, or consultation request data.

### Step 9

The terminal sends the user's input to the server as a new request for analysis or for reserving remote communication with a professional.

Input: User's inquiry, symptom description, or consultation request from Step 8.

Processing: The terminal serializes and transmits the input data securely to the server for further handling.

### Output: Input Data Delivered to the Server.

### Step 10

The server processes the user's input, generates a new prompt sentence incorporating user concerns or symptom descriptions, and submits it to the generative AI model. Based on the AI response, the server may offer a generated reply and available time slots for professional consultations.

Input: User's inquiry, symptom report, or consultation request from the terminal.

Processing: The server uses the generative AI model to generate context-appropriate advice or response and queries a scheduling system for available consultation slots.

Output: Generated response message and/or list of available professional appointment times.

### Step 11

The terminal receives the response message and available appointment options. The terminal then presents the response and scheduling interface to the user for confirmation.

Input: Response message and available appointment times from the server.

Processing: The terminal displays the AI-generated advice and appointment options in the user interface, enabling the user to confirm or choose a time slot.

Output: Advice and consultation scheduling options shown to the user.

### Step 12

The user selects and confirms a desired appointment time for remote consultation with a professional using the terminal's interface.

Input: List of available appointment times.

Processing: The user reviews options and interacts with the app to make a selection and confirm the booking.

Output: Confirmed appointment details ready to be communicated to the server.

### Step 13

The terminal transmits the user's booking selection to the server. The server then finalizes the reservation and distributes confirmation to both the user's terminal and the professional.

Input: User's confirmed appointment selection.

Processing: The server updates the reservation system, blocks the selected time slot, and notifies all parties.

Output: Confirmation notifications of the scheduled consultation provided to the user and the professional.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

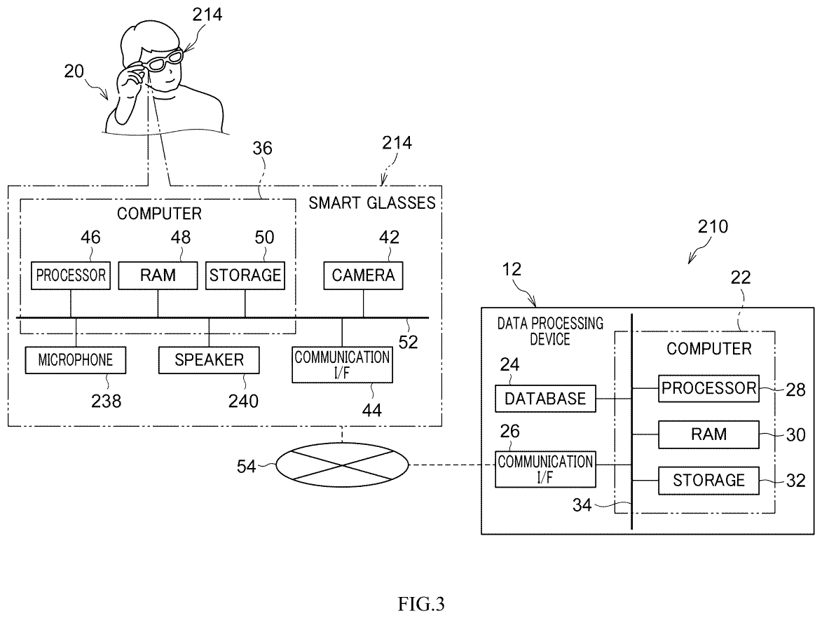

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

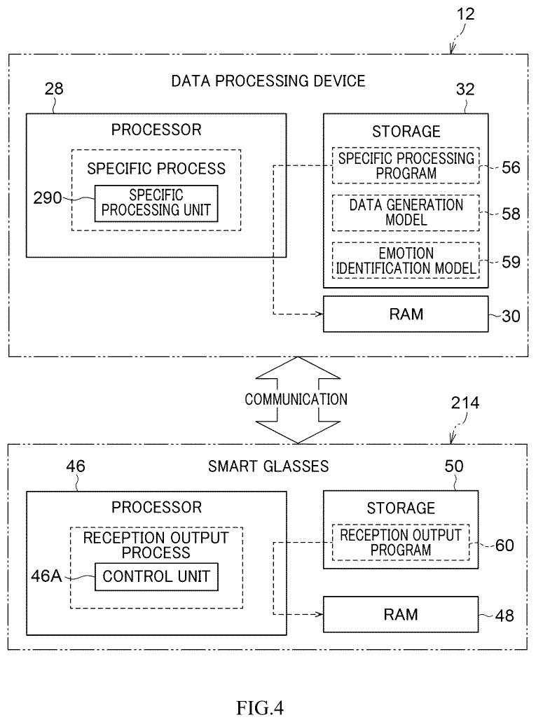

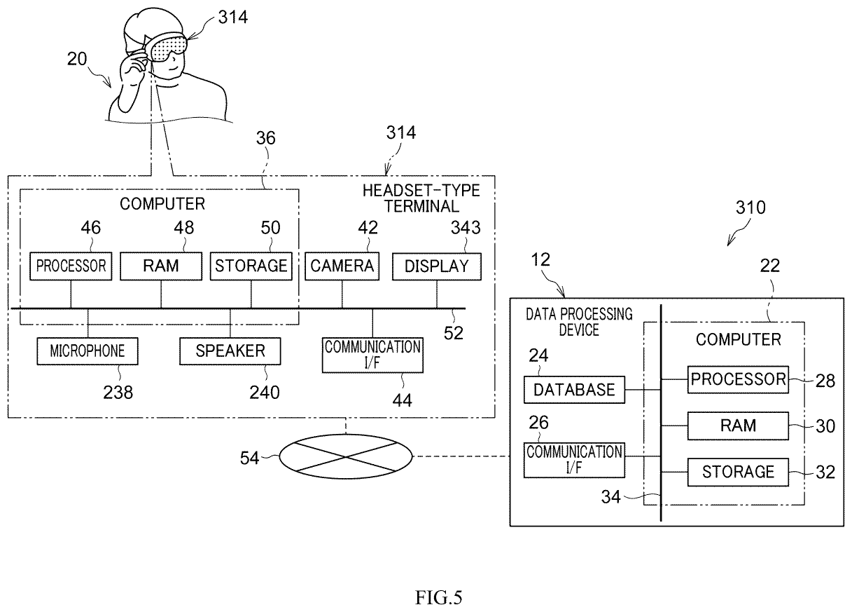

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples.

Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

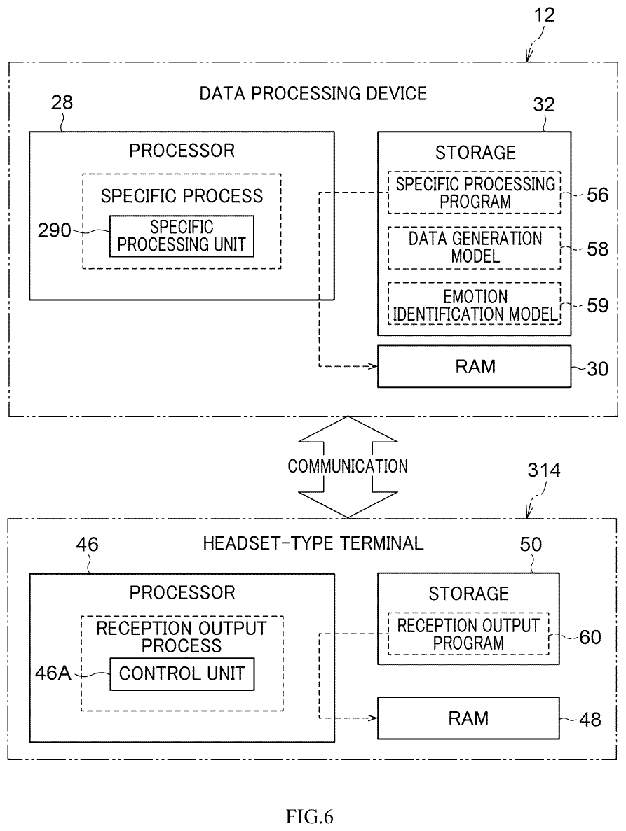

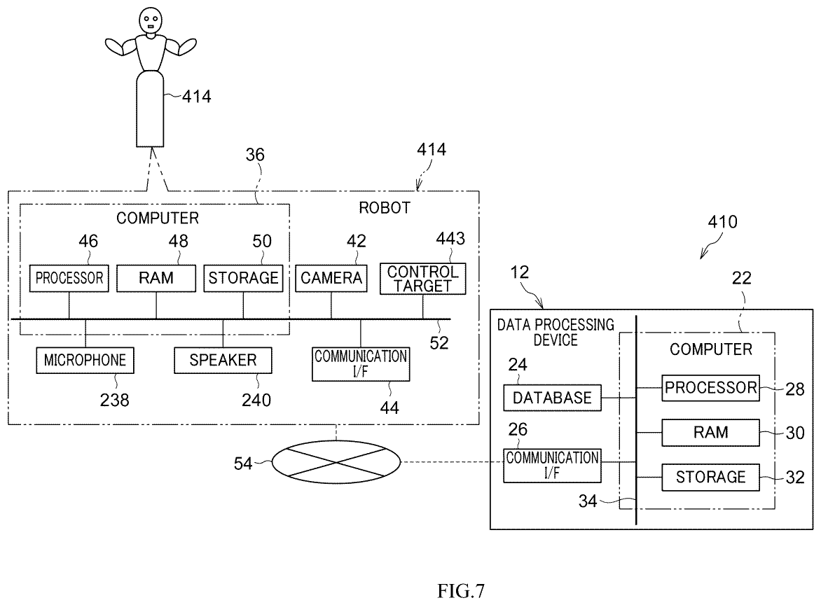

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

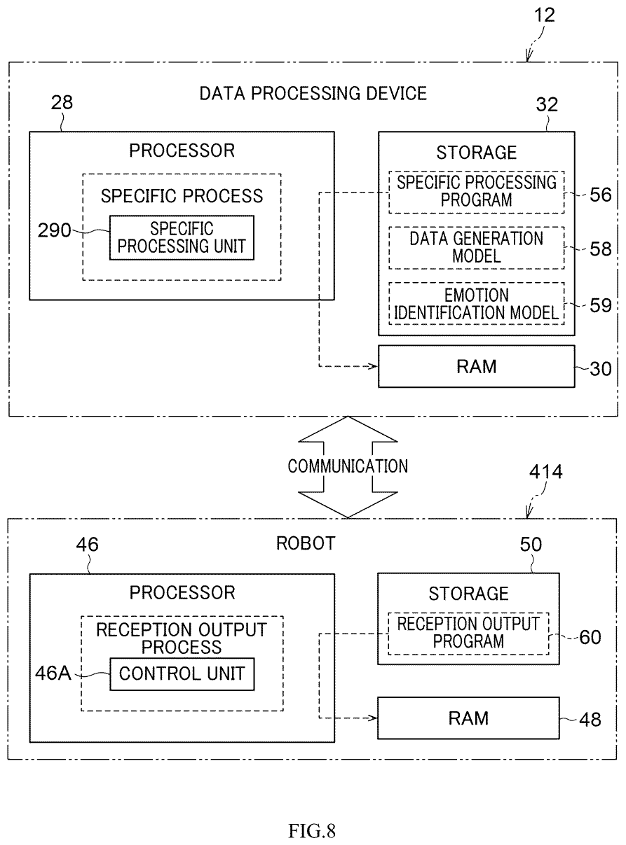

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

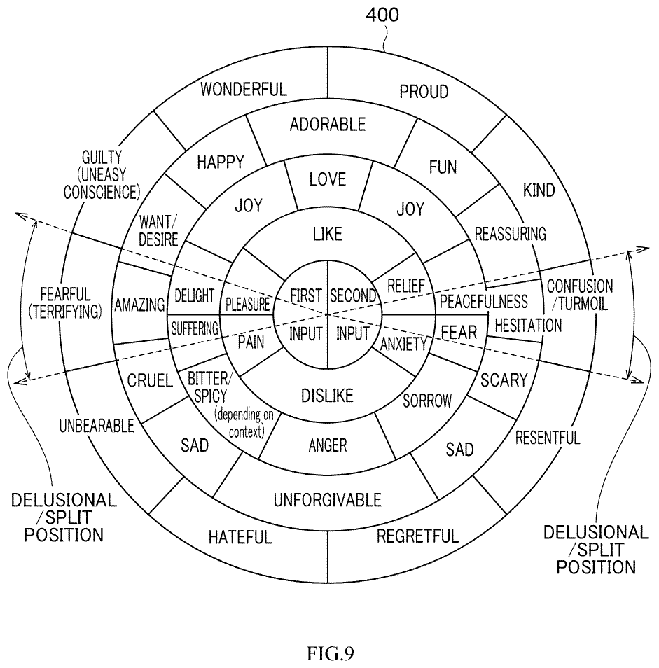

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more”and “want to know more”is experienced.

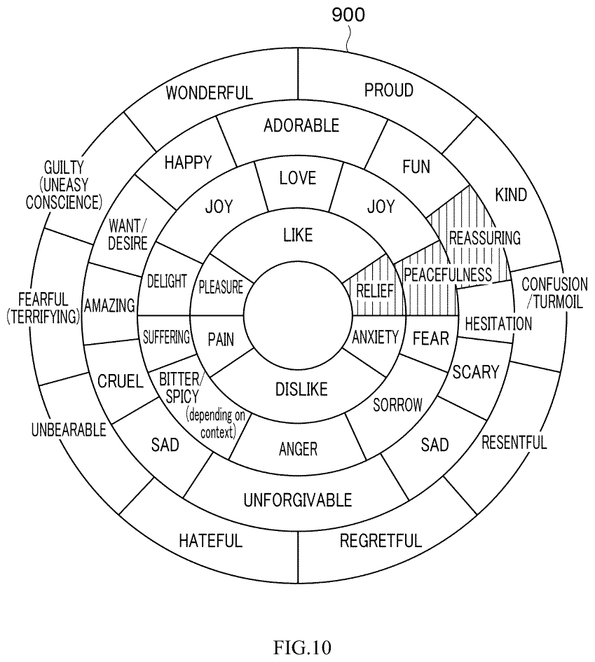

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor,
wherein the processor is configured to
acquire biological information of an animal from a biological information collection device,
transmit the acquired biological information to an information processing device,
input the acquired biological information into a generative artificial intelligence model and perform analysis processing,
wherein the analysis processing includes inputting a prompt sentence including an analysis instruction into the generative artificial intelligence model,
transmit results of the analysis processing to a user device and notify a user,
acquire input information or symptom-related information from the user, and generate response information using the generative artificial intelligence model,
transmit the generated response information to the user device and display it to the user,
provide remote consultation reservation with a specialist,
and integrally execute a series of processes by program control.

### Supplementary 2

The system according to supplementary 1,
wherein the processor is configured to
perform data preprocessing including outlier detection and missing value supplementation prior to analysis processing of the biological information.

### Supplementary 3

The system according to supplementary 1,
wherein the processor is configured to
identify abnormal values based on the biological information using the generative artificial intelligence model.

### Application Example 1

### Supplementary 1

A system comprising a processor,
wherein the processor is configured to
acquire behavioral information, rest information, and feeding information from an information acquisition device worn by an animal,
transmit the acquired information to a computing device,
input the acquired information into a generative artificial intelligence model and analyze the information,
transmit the analysis result to a display device and notify a user,
receive an inquiry or condition input relating to the animal from the user and generate advice information using the generative artificial intelligence model,
transmit the generated advice information to the display device and present it to the user,
provide a remote consultation reservation with a medical professional via a communication network,
recognize a user's emotional state and, based on the recognized emotional state, adjust the content or style of the advice information,
create a prompt sentence for input to a generative artificial intelligence model by combining the user's inquiry regarding the animal, the emotional state, and the acquired information, and use the prompt sentence to generate advice information,
generate period-based health analysis results based on behavioral information, rest information, and feeding information of the animal, and present the results to the display device.

### Supplementary 2

The system according to supplementary 1,
wherein the processor is configured to perform outlier detection and missing value imputation as preprocessing of the information.

### Supplementary 3

The system according to supplementary 1,
wherein the processor is configured to use the generative artificial intelligence model to identify abnormal values in the behavioral information, rest information, and feeding information.

### Example 2

### Supplementary 1

A system comprising a processor,
wherein the processor is configured to
collect biometric activity information from a biometric information collection device,
transmit the collected biometric activity information to a data processing apparatus,
perform, by the data processing apparatus, data preprocessing including outlier removal and missing value completion on the biometric activity information,
input the preprocessed biometric activity information into a generative information processing model and perform data analysis,
generate behavioral guidance information based on the analytical result by the data processing apparatus and transmit the behavioral guidance information to a terminal device for user notification,
receive input information regarding inquiries or biometric information from a user and generate response information by the generative information processing model,
transmit and display the response information to the terminal device, and
manage reservation for remote specialist communication.

### Supplementary2

The system according to supplementary 1,
wherein the processor is configured to
acquire emotional state information of the user using voice recognition and image recognition by the terminal device, and
analyze the emotional state information by the data processing apparatus and adjust the content of the behavioral guidance information based on the emotional state.

### Supplementary 3

The system according to supplementary 1,
wherein the processor is configured to
automatically generate a prompt sentence for the generative information processing model based on both biometric activity information and user emotional state information, and
generate the behavioral guidance information and the response information using the prompt sentence.

### Application Example 2

### Supplementary 1

A system comprising a processor,
wherein the processor is configured to
collect biological activity information, sleep information, and feeding information from a data collection device attached to an animal;
transmit the collected information to an information processing device via a communication apparatus;
obtain the collected information as well as emotion information of a user, and generate input data based on the collected information and the user's emotion information for use by a generative artificial intelligence model;
input the generated input data into the generative artificial intelligence model to perform analysis and generate advice information reflecting the user's emotional state based on an analysis result;
transmit the analysis result and the advice information to a terminal device and provide a notification and display to the user;
receive inquiry information or status information of the animal input from the user, and generate response information for the inquiry or the status information of the animal using the generative artificial intelligence model;
and transmit the generated response information to the terminal device for output to the user;
further provide a communication reservation with a professional.

### Supplementary 2

The system according to supplementary 1,
wherein the processor is configured to perform abnormal value determination processing or data completion processing as preprocessing on the collected information.

### Supplementary 3

The system according to supplementary 1,
wherein the processor is configured to perform abnormal value determination processing of the biological activity information, sleep information, and feeding information using the generative artificial intelligence model.

What is claimed is:

1. A system comprising a processor that is configured to:
collect data representing activity levels, sleep patterns, and food intake from an information collection device attached to a dog;
transmit the collected data to an analysis server, wherein the analysis server inputs the collected data into a generative artificial intelligence model and analyzes the data;
transmit analysis results to a terminal and notifies a user;
receive user input regarding questions or symptoms of the dog and generates advice using the generative artificial intelligence model;
transmit the generated advice to the terminal for display to the user; and
and provide online consultation reservation with a veterinarian.

2. The system according to claim 1, wherein the analysis server further preprocesses the data by filtering abnormal values and supplementing missing data.

3. The system according to claim 1, wherein the analysis server further identifies abnormalities in activity levels, sleep patterns, and food intake using the generative artificial intelligence model.
